(19) United States
(12) Patent Application Publication
(10) Pub. No.: US 2018/0359445 A1
(43) Pub. Date: Dec. 13, 2018

(54) Method for Recording Vehicle Driving Information and Creating Vehicle Record by Utilizing Digital Video Shooting

(71) Applicant: SanJet Technology Corp., Hsinchu (TW)

(72) Inventor: Yun-Song Liao, Hsinchu (TW)

(73) Assignee: SanJet Technology Corp., Hsinchu (TW)

(21) Appl. No.: 15/677,041

(22) Filed: Aug. 15, 2017

(30) Foreign Application Priority Data
Jun. 12, 2017 (TW) 106119528

(51) Int. Cl. H04N 5/76; G07C 5/00; H04N 5/38; G08G 1/04; B60W 50/00; A61B 5/00; A61B 5/11; A61B 5/18

(52) U.S. Cl. CPC H04N 5/76; G07C 5/004; H04N 5/38; G07C 5/008; G08G 1/04; H04L 67/10; A61B 5/0077; A61B 5/1118; A61B 5/18; A61B 5/0022; A61B 5/0013; B60W 50/0098

(57) ABSTRACT

A method for recording vehicle driving information and creating vehicle record by utilizing digital video shooting is provided. The method of the present invention is utilizing for transmitting driving video and driving behavior information provided by a vehicle driving video recording and analyzing device to a cloud server center for storing and analyzing. Here, by utilizing wireless transmission technology, the vehicle driving information, the driving video and the driving behavior information may be uploaded to the cloud server center for recording and big data analyzing, to generate travel log, driving information and vehicle driving history.

### BACKGROUND OF THE INVENTION

### Field of the Invention

The invention relates to a real-time driving recording system, and more particularly a method of utilizing digital video recording to record driving information and generate vehicle driving history, through cloud technology to transmit driving recording data and evaluate driving behavior.

### Description of Related Art

Currently, the countries of the world have major projects for researching in the technical field of intelligent transportation system. Especially, through analyzing the driver's driving behavior to survey the issue of driving safety, and the driving safety issue is derived from the driver's normal driving behavior and abnormal driving behavior. The current market has started selling the detection system of driving behavior for detecting the driver's driving behavior, and the method of detection includes analyzing physiological signals of the driver (for example, head movement, heart rate variability, wheel trajectory, eye movement).

According to the relevant literature on the analysis of driving behavior, it can be further classified from the analysis of signal sources, which can be divided into two categories. One is the physiological signal analysis method using driver physiological signals and their derived physiological states, and the other is the driving performance analysis method using the driver's performance as the analysis signal.

In addition to the analysis of driving behavior, a vehicle driving video recording device is needed to record the conditions encountered during driving. The general driving recorder only has photographic function and storage function. There is usually an emergency button on the driving recorder. When the button is pressed, the photographic information for a short period of time can be stored in a storage unit of the driving recorder, such as SD card. Generally, people use photographic function of driving recorder, and further make a video recording and storage. Then, the images or video information stored in the driving recorder is outputted or transferred to a computer, and a specific accident image may be segmented. Users can also manually upload the video content of the specific accident to the cloud network (such as youtube, FaceBook or cloud hard disk), by file transfer protocol software. In addition to keeping the vehicle driving condition, if there is a traffic dispute or an accident, the truth of the incident will be restored as soon as possible, and the responsibility for the accident will be clarified.

However, the disadvantages of the above approach include: cumbersome procedures of accessing image (video) file, need to remove the storage device (such as SD card) of the driving recorder and inserting into SD card slot of the computer to download the image (video) file and upload it to the cloud network, and consume a lot of awaiting time to download and upload images. Since the commonly uploaded image is only for individual to retain the daily driving record. If the user only displays the mobile (driving) images to his friends, the sharing of the mobile images is limited. In addition, if you upload mobile images to the Internet, you may violate other people's privacy due to exposing the license plate and face.

Furthermore, if the film shot by the driving recorder is not processed immediately, the stored images data in the driving recorder will be continuously cumulative over daily images recording. In addition to cause file arrangement inconvenience, the video recording also lost instantly sharing value of the road conditions. Although there have been using mobile phone immediately recording the traffic accident images, and the images are immediately uploaded to the cloud network (such as youtube, FaceBook or cloud disk). All the files uploaded to youtube, FaceBook and other related websites are not classified, so the traffic accident film is still difficult to find, and thus it ceases sharing value reduction.

In addition, the Taiwan patent number M462691 discloses an integrated driving recorder which includes a gravity sensing module for passive image capturing. The Taiwan patent No. 1538831 discloses a system and method of vehicle autonomous auxiliary driving, wherein the system consists of a device for detecting dangerous driving state, a vehicle auxiliary device and an electronic control unit, which can solve the issue that improving the driver and passenger safety driving assistant needs to set the expensive detection system, increasing the cost. The Taiwan patent No. M426792 discloses a driving recording device with lane identification ability, the architecture consists of a shooting module, an analysis module, an operation module, an alarm module, a display module, a GPS module and a storage module, in order to facilitate lane departure to the driver for warning and judgment. The Taiwan patent No. TW, 201616462A discloses a driving recorder with scoring function and its scoring method, and calculates a driving behavior score according to the result of comparison.

The invention improves the function of the existing driving recorder, and further proposes an invention with industrial utilization, and the invention has further advanced effects than prior arts; the description of present invention and implementation methods are described in detail in the following.

### SUMMARY OF THE INVENTION

The object of the present invention is to provide a method for recording driving information by using digital video shooting. Image recognition of a mobile recording and analysis device may be used to generate image frame or image data, and further analyzing image data to obtain a warning signal; utilizing driving behavior analysis (e.g. brake, mileage, lane departure, red lights, driving speed, acceleration, concentration degree), and transmitting the driving behavior analysis to the cloud server through wireless transmission, and also transmitting related data of the image recognition and the driving behavior to the cloud server. Based on the driving behavior analysis data in the cloud server, a big data analysis is performed by the cloud server, which can provide reference to other industries (such as: fleet management company, the insurance company), for example, evaluation of vehicle insurance, Usage Based Insurance (UBI). And, it may further provide vehicle resume.

The invention provides a method for recording driving information and generating a vehicle resume by using digital video recording. The system of the invention is used to transmit the mobile images and the driving behavior analysis data provided by the mobile recording and analysis device to the cloud server database by wireless technology. There are two main portions in this system, including a mobile recording and analysis device and a cloud server, in which the mobile recording and analysis device has a driving behavior analysis module, which is a part of the vehicle mounted device. The recording and analysis device includes an image capturing unit coupled to the first processing unit (including CPU, buffer, multiplexer and other processing unit) for capturing mobile images; a first wireless transmission module coupled to the first processing device for transmitting the mobile images and analysis data of driving behavior during a period of time. The cloud server includes a second processing unit, a UBI management module, a fleet management module and a second wireless transmission module to receive the mobiles images and the analysis data of driving behavior transmitted by the first wireless transmission module; the cloud server center is used to statistically calculate and comprehensively judge for the analysis data of driving behavior to obtain rating data of the driving behavior.

According to one aspect of the invention, the mobile recording and analysis device (such as driving recorder) is activating video shooting when the vehicle starts, and the video data is stored in the mobile recording and analysis device. The mobile recording and analysis device is provided with a button. When you press the button, the mobile images can be stored during a period of time (for example, as the vehicle accident is happened, the mobile images (video) data is also recorded and stored in the mobile recording and analysis device during the period of time).

The invention provides a method for recording vehicle driving information by digital video shooting, comprising: providing a mobile recording and analyzing device with an image capturing device and a first wireless transmission module; utilizing the image capturing device to capture moving images of a driving vehicle; based-on the moving images, recording driving track, road conditions and real scene to generate travel log and driving record; and transmitting the travel log and driving record to a cloud server center by the first wireless transmission module.

The method further comprising providing a gravity sensing module used to sense dynamic or static state of the vehicle to produce a gravity sensing value; and providing a global positioning system (GPS) for obtaining a location information corresponding to the vehicle. Based-on the location information, the moving images and the gravity sensing value, the driving behavior information is then generated.

The cloud server center includes a real-time information center and a data analyzing and processing center.

The first wireless transmission module is Bluetooth standard module, Wi-Fi wireless transmission standard module, 802.11x wireless transmission standard module, WiMAX (Worldwide Interoperability for Microwave Access) wireless transmission standard module.

The mobile recording and analyzing device includes a geographic database, a driving behavior information gathering system, an image encoding unit and a button.

A method for creating vehicle resume by digital video shooting, comprising: providing a mobile recording and analyzing device with an image capturing device and a first wireless transmission module; utilizing the image capturing device to capture moving images of a driving vehicle; based-on the moving images, utilizing a driving behavior information gathering system to create driving behavior information, and recording driving information of the driving vehicle; utilizing a classification unit to classify the driving behavior information to produce classification results; utilizing an evaluation unit to evaluate the classification results to generate evaluation results; and utilizing a conversion unit to translate the evaluation results into indexes of said vehicle resume.

The method further comprises transmitting the moving images, the driving information and the driving behavior information to a cloud server center by the wireless transmission module.

The method further comprises an analysis unit to analyze the driving behavior information to generate analysis results, and a statistical unit to statistically calculate the driving behavior information to generate statistical results.

The analysis unit, the statistical unit, the classification unit, the evaluation unit, and the conversion unit are configured in a driving behavior analysis system. The driving behavior analysis system is configured in a cloud server center.

### BRIEF DESCRIPTION OF THE DRAWINGS

The components, characteristics and advantages of the present invention may be understood by the detailed descriptions of the preferred embodiments outlined in the specification and the drawings attached:

FIG. 1 illustrates a system infrastructure diagram for insurance and fleet management by utilizing cloud driving recording information of the present invention;

FIG. 2 illustrates a vehicle-mounted device and a data analyzing and processing center according to one embodiment of the present invention;

FIG. 3 illustrates a mobile recording and analyzing device according to one embodiment of the present invention;

FIG. 4 illustrates a cloud server center according to one embodiment of the present invention;

FIG. 5 illustrates a driving behavior analysis system according to one embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

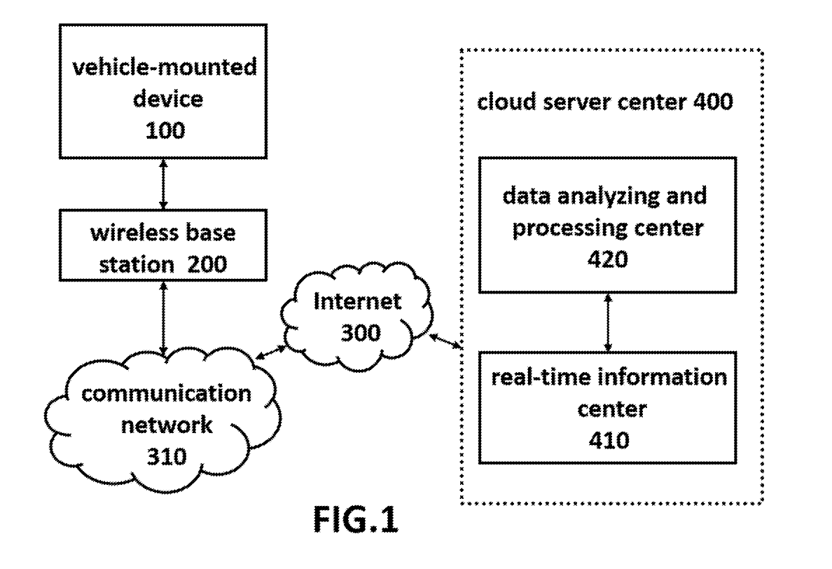

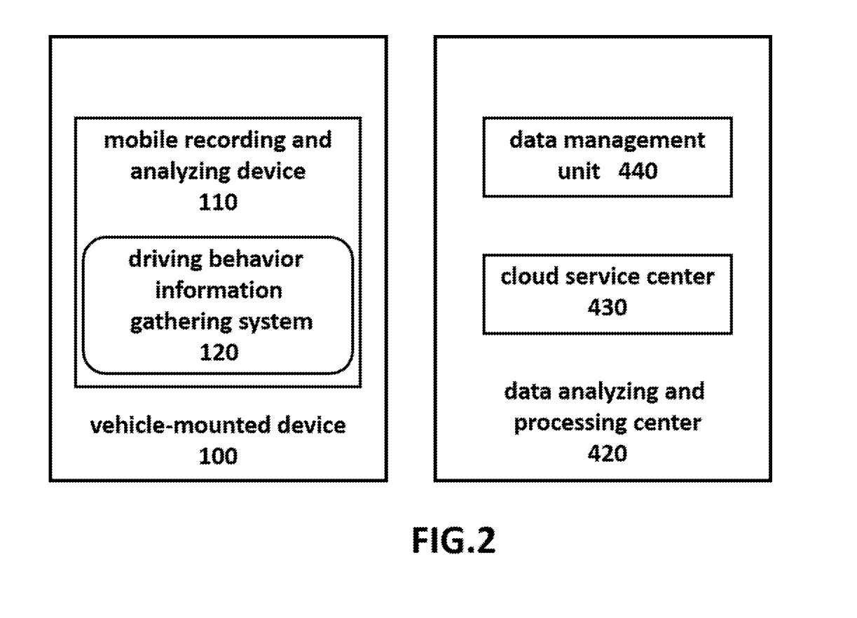

As shown in FIG. 1, it illustrates a system infrastructure diagram for insurance and fleet management by utilizing cloud driving recording information of the present invention. The system infrastructure for the insurance and fleet management by utilizing cloud driving recording information may include a vehicle-mounted device (on-board device) 100, a wireless base station 200, an Internet 300, a communication network (3G/4G/5G) 310 and a cloud server (center) 400. The vehicle-mounted device 100 is embedded/ disposed into a vehicle for driving control. In the example of a bus vehicle, the vehicle-mounted device 100 includes a hub coupled to multiple displays, a GPS module, a G-sensor, a driving behavior analysis device, a video playback unit (PIV & Video) and a mobile (images, video) recording and analyzing device. The vehicle-mounted device 100 includes the mobile recording and analyzing device 110 having a driving behavior information gathering system 120, and the cloud server center 400 includes a real-time information center 410 and a data analyzing and processing center 420, as shown in FIG. 2. The invention system with the cloud driving recording information is utilizing the vehicle-mounted device 100 to collect dynamic information of driving, such as mileage of driving, driving time, date, driving speed, engine speed, brakes, brake frequency, red lights, lane departure, spiritual concentration, acceleration, vehicle location and other driving information. The dynamic information (data) is processed by a digital encoding and an image data compressing, and then transmitting to the real-time information center 410 of the cloud server center 400 through the wireless base station 200, the communication network 310 and the Internet 300. The real-time information center 410 is for example driving monitoring center, such as a computer or a server. The real-time information center 410 is mainly used to record the driving information, providing for the third party capable of real-time browsing real-time information of driving and sending to the data analyzing and processing center 420 of the cloud server center 400 for retrieving, analyzing and evaluating the driver information of a vehicle driver. In one embodiment, the data analyzing processing center 420 includes a cloud service center 430 and a data management unit 440. For example, functions of the data management unit 440 include vehicles management, terminal stops management, routes management, users management and responsibility management, and functions of the cloud service center 430 include analysis of driving behavior, analysis of traffic or road conditions, smartly monitoring, insurance valuation and fares tracking and management. The mobile recording and analyzing device 110 may provide images of specific sections, traffic conditions, or road conditions.

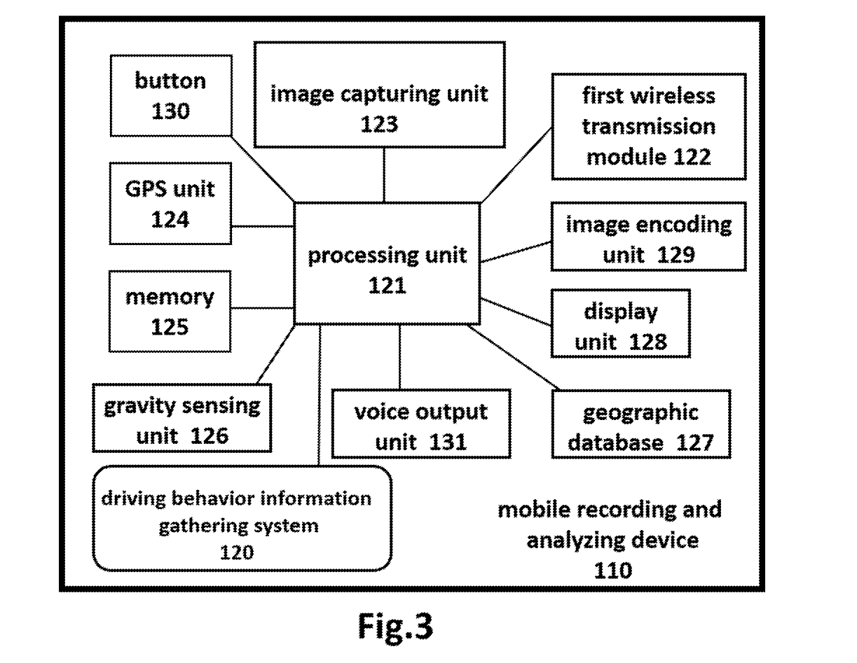

As shown in FIG. 3, it illustrates a functional block diagram of the mobile recording and analyzing device 110 according to an embodiment of the present invention. The mobile recording and analyzing device 110 comprises a driving behavior information gathering system 120, a processing unit 121, an image capturing unit 123, a first wireless transmission module 122, a display unit 128, an image encoding unit 129, a voice output unit 131, a button 130 and a memory 125. The image capturing unit 123 may be used to acquire continuous images in vehicle driving. The voice output unit 131 may be optionally configured in the mobile recording and analyzing device 110. In this embodiment, by utilizing the cloud driving recording information system of the invention, mobile images and data of analyzing driving behavior may be automatically wirelessly transmitted to the wireless base station 200 by the mobile recording and analyzing device 110, and followed by automatically uploading to the cloud server center 400 through the communication network 310 and the Internet 300 such that the remote server can instantly share real-time images and analysis data of driving behavior, and further performing big data statistical analysis and evaluation of driving behavior.

The memory 125 includes ROM, RAM or non-volatile flash memory.

As shown in FIG. 3, the mobile recording and analyzing device 110 includes a button 130. When the button 130 is pressed, the image capturing unit 123 coupled to the processing unit 121 can retrieve moving images for a period of time. The moving images can be stored in the memory 125. In other words, the mobile recording and analyzing device 110 can provide information such as photographs, image recognition, driving recording video, driving behavior analysis data, etc.. The first wireless transmission module 122 (such as WiFi module) is coupled to the wireless base station 200, and communicates with the communication network 310, the Internet 300 and the second wireless transmission module 403 of the cloud server center 400. Therefore, the cloud server center 400 receives the photographs, the image recognition, the driving recording video, the driving behavior analysis data from the first wireless transmission module 122. In case of an accident, the photographs or images received from the cloud server center 400 can be used as evidence of the accident. When the moving images are transmitted to the cloud server center 400 and processed by the central processing unit 401, it is stored in a memory 412 coupled to the central processing unit 401. In one embodiment, the display unit 128 is used to display a warning message, and the display unit comprises a liquid crystal display or a touch screen, or a head up display (HUD). In one embodiment, the mobile recording and analyzing device 110 further includes a voice playback unit (e.g., a speaker) to play a warning message.

In another embodiment, the vehicle-mounted device 100 further includes an advanced driver assistance systems (ADAS) which may provide relevant information, such as working conditions of the vehicle, environmental changes outside the vehicle to be analyzed, for the vehicle driver. For example, ADAS includes Blind Spot Detection System, Backup Parking Aid System, Rear Crash Collision Warning System, Forward Collision Warning System, Lane Departure Warning System, Collision Mitigation System, Adaptive Front-lighting System, Night Vision System, Adaptive Cruise Control System, Pre Crash System and Parking Aid System.

As shown in FIG. 3, the mobile recording and analyzing device 110 of the present invention includes a driving behavior information gathering system 120. The image capturing unit 123 captures the images, and the mobile recording and analyzing device 110 immediately performs high-definition images recording and images recognition. Later, based on the recorded images and the images recognition data, the driver behavior information gathering system 120 performs a driving behavior analysis to generate driving behavior related data. Similarly, through wireless communication and/or Internet, the driving behavior related information will be transmitted to the remote server, through the wireless transmission module 122, such as wireless LAN communication module. The first wireless transmission module 122 is coupled to the processing unit 121. For example, the first wireless transmission module 122 is WiFi module. The dynamic driving data is digitally encoded and compressed, and then transmitted via the first wireless transmission module 122.

As shown in FIG. 3, the mobile recording and analyzing device 110 further includes a global positioning system (GPS) 124, a geographic database 127 and a gravity sensing unit (G-sensor) 126. The GPS 124 is provided for obtaining a location information corresponding to the vehicle, and the geographic database 127 is used to store a map information and a map location information corresponding to the map information, wherein the map location information is compared with the location information to search map information corresponding to location information. The gravity sensing unit 126 is used to sense dynamic or static state of the vehicle to produce a gravity sensing value. In addition, the processing unit 121 is coupled (electrically connected) to the GPS 124, the geographic database 127, the image capturing unit 123 and the gravity sensing unit (G-sensor) 126. Based-on the location information, continuous images and gravity sensing value, the detection and analysis of the driving behavior are made, and thereby calculating the driving behavior information. In one embodiment, the GPS 124, the geographic database 127 and the gravity sensing unit (G-sensor) 126 are integrated into the driving behavior information gathering system 120. Thus, the driving behavior information gathering system 120 includes the GPS 124, the geographic database 127 and the gravity sensing unit (G-sensor) 126. Then, the first wireless transmission module 122 (such as WiFi module) is coupled to the wireless base station 200, and establishes a connection with the second wireless transmission module 403 of the cloud server center 400 through the communication network 310 and the Internet 300. Therefore, the cloud server center 400 receives analyzed data or information of the driving behavior transmitted from the first wireless transmission module 122. Then, based-on the received lot of driving behavior data (big data) transmitted from the first wireless transmission module 122, statistical analysis and comprehensive judgment of big data are performed in the cloud server center 400, in order to get the rating scales data or information of the driving behavior to evaluate insurance of driver or vehicle, or the fare.

In one embodiment, the image capturing unit is photographed toward one field of view of the vehicle in order to form a vehicle exterior image. In one embodiment, the memory 125 is used to store the location information, the continuous images, the gravity sensing values and the calculated rating scales data of the driving behavior. The gravity sensing unit 126 is used for sensing dynamic or static state of the vehicle, to create a gravity sensing value, and thereby detecting whether the driver does overspeed behavior or not. The processing unit 121 is coupled (electrically connected) to the GPS 124, the geographic database 127, the image capturing unit 123 and the gravity sensing unit (G-sensor) 126, the display unit 128 and the speaker. Based-on the location information, continuous images and gravity sensing acceleration value, detection and analysis of the driving behavior are made, and rating scales data of the driving behavior is then calculated.

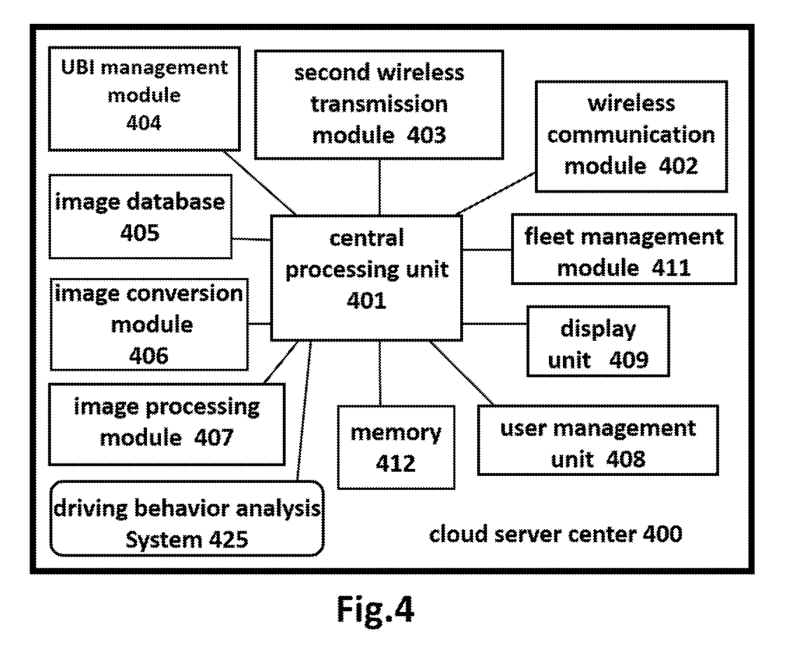

As shown in FIG. 4, it illustrates a functional block diagram of the cloud server center 400 of the present invention. The cloud server center 400 includes a central processing unit 401, a wireless communication module 402, a second wireless transmission module (LAN communication module) 403, a usage based insurance (UBI) management module 404, an image database 405, an image conversion module 406, an image processing module 407, a user management unit 408, a display unit 409, a fleet management module (or system) 411, a memory 412 and a driving behavior analysis system 425. The wireless communication module 402, the second wireless transmission 403, the UBI management module 404, the image database 405, the image conversion module 406, the image processing module 407, the user management unit 408, the display unit 409, the fleet management module (or system) 411, the memory 412 and the driving behavior analysis system 425 are coupled to the central processing unit 401. The image conversion module 406 is used to process moving images and change the format of the moving images. The image database 405 is used to store the moving images converted by the image conversion module 406. Through the wireless communication module 402 of the cloud server center 400 of the invention and/or the second wireless LAN communication module 403, the moving images and driving behavior data of the mobile recording and analyzing device 110 will be automatically transmitted to the cloud server center 400, and the cloud server center 400 can instantly share real-time images and analyze and evaluate the driving behavior. In one embodiment, the driving behavior is analyzed by the driving behavior information gathering system 120 to produce the driving behavior data. The driving behavior data (big data) is transmitted to the cloud server center 400 for statistically analyzing of big data, to make an evaluation on the driving behavior. According to judgment and evaluation results of the driving behavior information, they are provided to the UBI management module 404 and the fleet management module 411 as an evaluation of insurance for the driver or the vehicle, or fare assessment. For example, the fleet management module (or system) 411 includes fleet management or fare management.

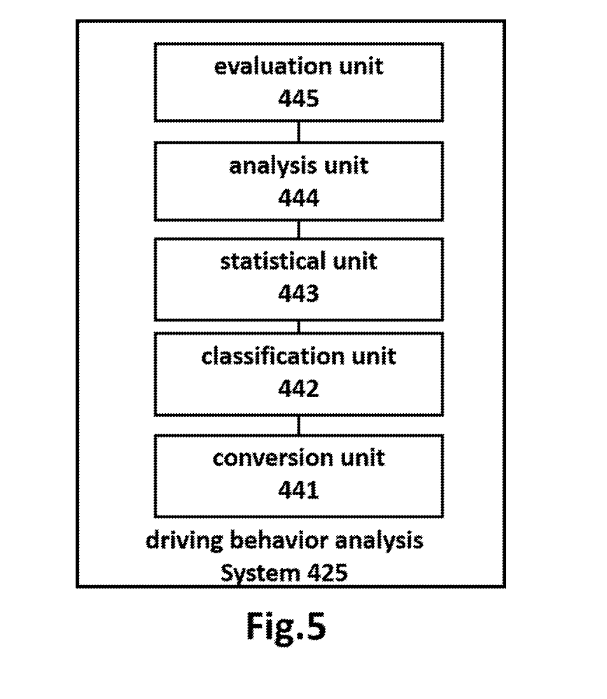

In one embodiment, the driving behavior analysis system 425 includes a conversion unit 441, a classification unit 442, a statistical unit 443, an analysis unit 444, and an evaluation unit 445, as shown in FIG. 5. In another embodiment, an on-board diagnostics (OBD) system can be integrated into the driver behavior information gathering system 120.

When a driver operates to drive a vehicle, driving behavior data and driving related data can be generated. For example, the driving information includes mileage of driving, date, driving time, driving speed, acceleration, driving location or change of tilt angle of the vehicle, brake, emergency brake, sharp bend, lane departure, forward collision, car accident, throttle depth, energy consumption and engine rotational speed. The classification unit 442 can be used to classify the driving behavior information (data) to produce classification results of each parameter (driving parameter). The statistical unit 443 may statistically calculate the driving behavior data to create statistical results of each parameter (driving parameter). The analysis unit 444 can be used to analyze the driving behavior data to produce the analysis results of each parameter (driving parameter). Then, the evaluation unit 445 is used to evaluate the results of the above-mentioned classification, statistics and analysis in order to generate evaluation results. The conversion unit 441 is used to translate the evaluation results generated by the evaluation unit 445 into the indexes of the vehicle resume.

In one embodiment, the classification unit 442 includes a driving behavior definition which makes the driving behavior data divide into a plurality of behavior categories, including initial start behavior, parking behavior, acceleration behavior, deceleration behavior, sliding behavior, idle behavior, emergency brake behavior, sharp bend behavior, lane departure behavior, forward collision behavior, stably driving behavior and so on. According to the results of the classification, statistics and analysis, the evaluation unit 445 is used for evaluating the driver's driving behavior, including initial start behavior, parking behavior, acceleration behavior, deceleration behavior, sliding behavior, idle behavior, emergency brake behavior, sharp bend behavior, lane departure behavior, forward collision behavior and stably driving behavior . . . etc, to generate evaluation results. For example, the evaluation results include: the proportion of all kinds of driving behaviors, frequency (the number of times), or evaluation of extent for each driving behavior in one driving road trip or driving process (such as, from start location to destination location), wherein the driving behavior includes but not limited to initial start behavior, parking behavior, acceleration behavior, deceleration behavior, sliding behavior, idle behavior, emergency brake behavior, sharp bend behavior, lane departure behavior, forward collision behavior and stably driving behavior. The conversion unit 441 is used to translate the evaluation results generated by the evaluation unit 445 into the indexes of the vehicle resume or the rating scales data. These indexes can be defined by scores or grades.

Based-on the driving behavior and driving information recorded and analyzed by the vehicle-mounted device (on-board device), the events or situations of driving can be scored accordingly during the driving process. For example, when the continuous image captured by the image capturing unit is analyzed, the score can be adjusted as the driving vehicle does not keep a safe distance from the front vehicle. The method of adjustment can be carried out by means of adding score, subtracting score or averaging some scores.

That is, in the present invention, the conversion unit 441 converts the evaluation results generated by the evaluation unit 445 to reference indexes, based on the driving information and the reference indexes to generate a vehicle resume.

It is noted that the purpose of the invention is not to analyze driver behavior itself, but analyzing the driving behavior and utilizing analysis or statistics of the driving information to complete the vehicle resume. The meaning of the vehicle's resume is related to all driving records of previously driving the vehicle, including all driving information records as well as the evaluated and converted reference indexes. The content of the vehicle resume can serve as a reference or calculation of the residual value of the vehicle, or an objective assessment of the value of the vehicle, and therefore preserving the value of the vehicle. The vehicle owner can also be indirectly encouraged to install the vehicle-mounted device of the invention.

The wireless local area network communication module (WLAN module) 122 is compatible with the wireless LAN protocol to facilitate data transmission. For example, the wireless LAN communication module 122 is compatible with Bluetooth standard module, Wi-Fi wireless transmission standard module, 802.11x wireless transmission standard module, WiMAX (Worldwide Interoperability for Microwave Access) wireless transmission standard module. Using the wireless LAN communication module 122, the vehicle-mounted device (on-board device) 100 of the present invention can synchronously wirelessly transmitting the image signal of driving or score data of the driving behavior to the cloud server center 400.

In one embodiment, the driving behavior analysis system 425 is configured in the cloud server center 400, electrically coupled to the central processing unit 401. That is, the production of the vehicle resume (history) is performed in the driver behavior analysis system 400 of the cloud server center 425.

As noted above, the features of the invention at least include:

1. using Driving Video Recording (DVR) platform or system to record real scene, driving track and road conditions, GPS module, G-sensor and image capturing device (camera) built in the DVR platform to generate video (visual) images, and based-on the recorded driving track, road conditions and real scene to generate travel log and driving record which may be stored in the cloud server for sharing;

2. using DVR platform to create vehicle resume, which mainly uses (a) recording driving information (time, distance, track, road conditions and real scene), and (b) analyzing driving behavior, utilizing the combination of the (a) and (b) to produce the vehicle resume, which can also analyze, compute, store and share the vehicle resume by the cloud server; the vehicle resume can be used as the basis for evaluating the value of the vehicle, or rates of insurance or loan.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. A method for recording vehicle driving information by digital video shooting, comprising:
providing a mobile recording and analyzing device with an image capturing device and a first wireless transmission module;
utilizing said image capturing device to capture moving images of a driving vehicle;
based-on said moving images, recording driving track, road conditions and real scene to generate travel log and driving record; and
transmitting said travel log and driving record to a cloud server center by said first wireless transmission module.

2. The method of claim 1, further comprising providing a gravity sensing module used to sense dynamic or static state of said vehicle to produce a gravity sensing value.

3. The method of claim 2, further comprising providing a global positioning system (GPS) for obtaining a location information corresponding to said vehicle.

4. The method of claim 3, based-on said location information, said moving images and said gravity sensing value to generating said driving behavior information.

5. The method of claim 1, wherein said cloud server center includes a real-time information center and a data analyzing and processing center.

6. The method of claim 1, wherein said first wireless transmission module is Bluetooth standard module, Wi-Fi wireless transmission standard module, 802.11x wireless transmission standard module, WiMAX (Worldwide Interoperability for Microwave Access) wireless transmission standard module.

7. The method of claim 1, wherein said mobile recording and analyzing device includes a geographic database.

8. The method of claim 1, wherein said mobile recording and analyzing device includes a driving behavior information gathering system.

9. The method of claim 1, wherein said mobile recording and analyzing device includes an image encoding unit.

10. The method of claim 1, wherein said mobile recording and analyzing device includes a button.

11. A method for creating vehicle resume by digital video shooting, comprising:
providing a mobile recording and analyzing device with an image capturing device and a first wireless transmission module;
utilizing said image capturing device to capture moving images of a driving vehicle;
based-on said moving images, utilizing a driving behavior information gathering system to create driving behavior information, and recording driving information of said driving vehicle;
utilizing a classification unit to classify said driving behavior information to produce classification results;
utilizing an evaluation unit to evaluate said classification results to generate evaluation results; and
utilizing a conversion unit to translate said evaluation results into indexes of said vehicle resume.

12. The method of claim 11, further comprising transmitting said moving images, said driving information and said driving behavior information to a cloud server center by said wireless transmission module.

13. The method of claim 11, further comprising an analysis unit to analyze said driving behavior information to generate analysis results.

14. The method of claim 12, further comprising a statistical unit to statistically calculate said driving behavior information to generate statistical results.

15. The method of claim 14, wherein said analysis unit, said statistical unit, said classification unit, said evaluation unit and said conversion unit are configured in a driving behavior analysis system.

16. The method of claim 15, wherein said driving behavior analysis system is configured in a cloud server center.

17. The method of claim 11, wherein said mobile recording and analyzing device includes a geographic database.

18. The method of claim 11, wherein said mobile recording and analyzing device includes said driving behavior information gathering system.

19. The method of claim 11, further comprising providing a gravity sensing module used to sense dynamic or static state of said vehicle to produce a gravity sensing value, and a global positioning system (GPS) for obtaining a location information corresponding to said vehicle.

20. The method of claim 19, based-on said location information, said moving images and said gravity sensing value to generating said driving behavior information.
